(19) United States
(12) Patent Application Publication
(10) Pub. No.: US 2010/0056954 A1
(43) Pub. Date: Mar. 4, 2010

(54) Device For Extracting Blood Samples

(76) Inventor: Eli Oren, Ramat Hasharon (IL)

Correspondence Address: The Law Office of Michael E. Kondoudis, 888 16th Street, N.W., Suite 800, Washington, DC, 20006, US

(21) Appl. No.: 12/416,539

(22) Filed: Apr. 1, 2009

Related U.S. Application Data
(60) Provisional application No. 61/190,594, filed on Sep. 2, 2008.

(51) Int. Cl. A61B 5/15

(52) U.S. Cl. 600/573

(57) ABSTRACT

A device for extracting blood samples comprising a casing, a cover, a suction device, and a pricking element. The casing exhibits a finger opening and a suction opening and comprises a container exhibiting a sealable sample opening and posited below the finger opening. The cover is sealably connected to the casing and seals the sample opening. The suction device is sealably connected to the suction opening of the casing. In operation, the casing is sealed by placing a finger upon the finger opening, the suction device is arranged to generate a partial vacuum. The pricking element is arranged to prick the finger from within the casing through the finger opening such as to cause blood to trickle from the pricked finger. The partial vacuum enhances blood trickling from the pricked finger. The container holds the blood sample that may be removed upon releasing the partial vacuum.

### CROSS REFERENCE TO RELATED APPLICATIONS

This application claims the benefit of U.S. Provisional Patent Application 61/190,594 filed on Sep. 2, 2008, which is incorporated herein by reference.

### BACKGROUND

1. Technical Field

The present invention relates to the field of medical appliances, and more particularly, to a blood sampler.

2. Discussion of Related Art

Blood samples are required for a wide range of medical applications and treating processes. These include treatment which patients must apply themselves according to measured blood values and various diagnostic procedure that require a quick and reliable blood sampling. Blood sampling also requires a high level of hygiene that is not ensured by many of the current methods.

The following documents are incorporated herein by reference in their entirety: WIPO Publication No. WO2005120351, which discloses a vacuum sample expression device; U.S. Patent Publication No. US2007016239, which discloses a lancing device, method of making lancing device, pump mechanism, and sucking device; U.S. Patent Publication No. US2001031931, which discloses a method and apparatus for obtaining blood for diagnostic tests; U.S. Pat. No. 6,206,841, which discloses a method and apparatus for obtaining blood for diagnostic tests; and U.S. Pat. No. 5,320,607, which discloses a simple blood sampling device.

Available blood sampling instruments are designed for recurrent use, and thus require sterilization and lancet replacement between uses. Furthermore, available blood sampling instruments utilize a power source to activate a pump that generates the partial vacuum.

### BRIEF SUMMARY

Embodiments of the present invention provide a device for extracting blood samples. One device comprises a casing, a cover, a suction device, and a pricking element. The casing exhibits a finger opening and a suction opening. The casing comprises a container exhibiting a sealable sample opening and posited below the finger opening. The cover is sealably connected to the casing such that the cover operably seals the sample opening. The suction device is sealably connected to the suction opening of the casing. The casing is operably sealable by placing a finger upon the finger opening. The suction device is arranged to operably generate a partial vacuum characterized by an air pressure lower than atmospheric pressure, in the sealed casing. The pricking element is arranged to prick the finger from within the casing through the finger opening such as to cause blood to trickle from the pricked finger. The partial vacuum enhances blood trickling from the pricked finger, and the air pressure is selected to enhance blood trickling. The container is arranged to hold a blood sample from the pricked finger. The sample opening is arranged to allow the blood sample through upon releasing the partial vacuum.

Accordingly, according to an aspect of the present invention, there is provided a device, wherein the pricking element comprises: a lever pivotly connected to the casing, exhibiting an inner arm substantially inside the casing and an outer arm substantially outside the casing; and a pricker mounted on the inner arm of the lever. The lever is arranged to bring the pricker in contact with the finger upon pushing the outer arm, and the pricker is arranged to prick the finger.

Accordingly, according to another aspect of the present invention, there is provided a device, wherein the suction device comprises a piston arranged to reduce air pressure in the sealed casing upon expansion.

Accordingly, according to yet another aspect of the present invention, there is provided a device that is ready to use, disposable, sterilized and easy to operate independently of a power source.

Embodiments of the present invention provide a method of extracting blood samples. One method comprises: placing a finger on an opening in an otherwise sealed pricking device, thereby rendering the pricking device airtight; generating a partial vacuum in the sealed pricking device; pricking the finger from within the sealed pricking device through the opening, thereby causing blood to trickle from the finger; collecting a blood sample from the trickling blood; and removing the partial vacuum and thereby allowing removing finger and removing the blood sample. The partial vacuum is selected such as to enhance blood trickling.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

The present invention will be more readily understood from the detailed description of embodiments thereof made in conjunction with the accompanying drawings of which:

FIGS. 1A, 1B and 1C are schematic cross section illustrations of a device for extracting blood samples, according to some embodiments of the invention; and

FIG. 2 is a high level schematic flowchart a method of extracting blood samples, according to some embodiments of the invention.

### DETAILED DESCRIPTION

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

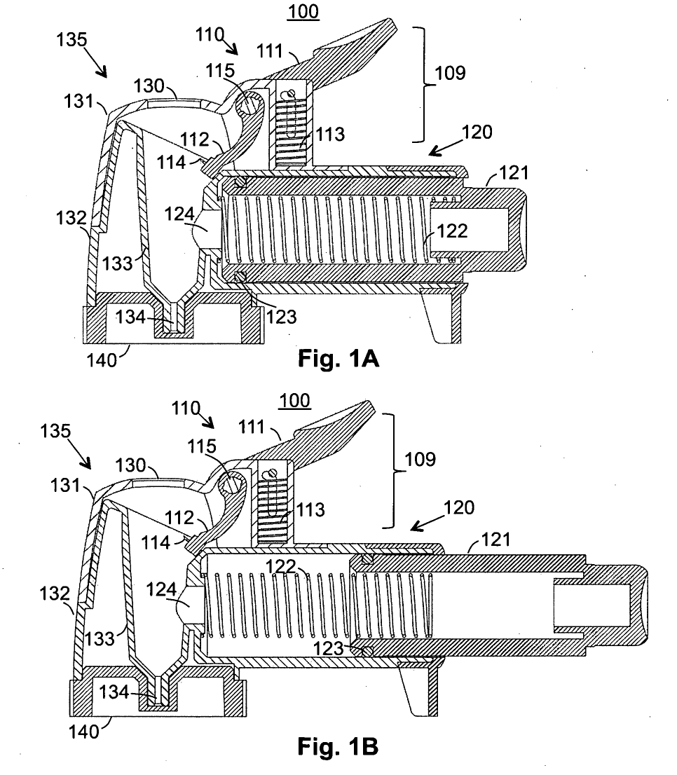

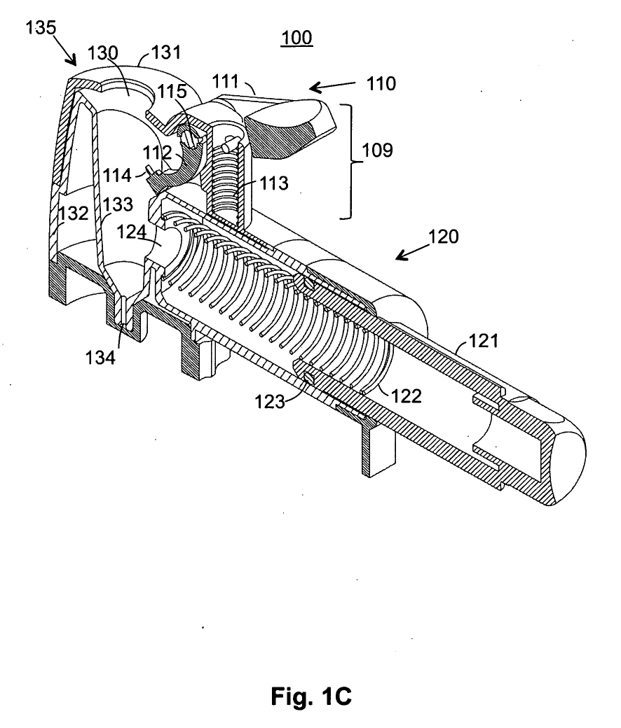

FIGS. 1A, 1B and 1C are schematic cross section illustrations of a device 100 for extracting blood samples, according to some embodiments of the invention. FIG. 1C is a perspective cross section. Device 100 comprises a casing 135, a container 133, a cover 140, a suction device 120, and a pricking element 109. Casing 135 exhibits a finger opening 130 and a suction opening 124. Casing 135 comprises a container 133 exhibiting a sealable sample opening 134 and posited below finger opening 130. Container 133 may be integrated within casing 135. Casing 135 may comprise a lower part 132 comprising container 133 and suction device 120 and an upper part 131 comprising finger opening 130 and pricking element 109, upper part 131 is sealed upon lower part 132. Cover 140 is sealably connected to casing 135 such that cover 140 operably seals sample opening 134. After operating device 100, cover 140 may be removed from casing 135 (e.g., cover 140 may be screwed off casing 135), and allow removing the blood sample from container 133. Suction device 120 is sealably connected to suction opening 124 of casing 135. Suction device 120 is arranged to operably generate a partial vacuum in sealed casing 135. Suction device 120 may implement a manual suction mechanism. For example, suction device 120 may comprise a piston 121. Pulling piston 121 may reduce air pressure in sealed casing 135 due to an expansion of its volume (FIGS. 1B, 1C). Piston 121 may be arranged to release the partial vacuum upon contraction. Piston 121 may be connected to a piston spring 122 arranged to hold piston 121 in a contracted state (FIG. 1A). Piston 121 may be sealed by sealing means 123, e.g., an O ring.

According to some embodiments of the invention, pricking element 109 comprises a lever 110 pivotly connected to casing 135 and which pivots about pivot 115. Lever 110 exhibits an inner arm 112 substantially inside casing 135 and an outer arm 111 substantially outside casing 135. A pricker 114 is mounted on inner arm 112 of lever 110. Lever 110 is arranged to bring pricker 114 in contact with the finger upon pushing outer arm 111 from outside casing 135. Outer arm 111 of lever 110 may be connected to a lever spring 113 arranged to remove pricker 114 from the finger upon releasing outer arm 111.

In operation, casing 135 is sealed by placing a finger (not shown) upon finger opening 130 (suction opening 124 is sealed by suction device 120 and sample opening 134 is sealed by cover 140). Then, suction device 120 is applied to generate a partial vacuum in sealed casing 135 (e.g., by reducing air pressure in sealed casing 135 below the atmospheric pressure, due to an expansion of its volume, as illustrated in FIGS. 1B, 1C). Pricking element 109 is arranged to prick the finger from within casing 135 through finger opening 130 such as to cause blood to trickle from the pricked finger. For example, when pricking element 109 comprises lever 110 with pricker 114 mounted upon inner arm 112, lever 110 is applied to bring pricker 114 in contact with the finger upon pushing outer arm 111. Pricker 114 is arranged to prick the finger, and container 133 is arranged to hold a blood sample from the pricked finger. The blood sample trickles from the punctured finger due to the partial pressure and into container 133 posited below finger opening 130. The partial vacuum enhances blood trickling from the pricked finger, and the air pressure (below atmospheric pressure) is selected to enhance blood trickling. Finally, the blood sample is removed from container 133 through sample opening 134, which is arranged to allow the blood sample through upon releasing the partial vacuum (e.g., by increasing air pressure back to atmospheric pressure using suction device 120, such as by contracting piston 121). Releasing the partial vacuum may be carried out by contracting suction device 120 (e.g., pushing in piston 121) and thereby increasing air pressure back to atmospheric pressure. The rebuilt pressure may allow releasing the finger which is operably fixated to finger opening 130 by the partial vacuum, and allow release of the blood sample upon removing (e.g., unscrewing) cover 140.

According to some embodiments of the invention, device 100 may further comprise a cap (not shown) arranged to seal finger opening 130. The cap may either keep device 100 sterile till use, protect against accidental application of pricking element 110, keep the extracted blood sample in sterile conditions till it is further analyzed or used, or a combination of the above.

According to some embodiments of the invention, device 100 may further comprise a medical testing means (not shown) integrated within device 100. The medical testing means may be integrated in device 100, e.g., in cover 140, in sample opening 134, in container 133 itself. The medical testing means may be integrated within casing 135 such that it and its results are visible from outside casing 135, e.g., through a transparent part in casing 135.

According to some embodiments of the invention, suction device 120 may be further arranged to allow generating a predefined pressure, and container 133 may be further arranged to allow removing the blood sample from container 133 through sample opening 134 upon the generation of the predefined pressure. In this way, suction device 120 may be arranged to allow exerting the blood sample from device 100 in a controllable manner using the predefined pressure application.

According to some embodiments of the invention, suction device 120 may be further arranged to allow controlling an amount of blood trickling from the pricked finger by regulating the air pressure and a time of using device 100. Due to the possibility to adjust the pressure inside casing 135 by using the manual suction mechanism of suction device 120, the amount (e.g., volume) of the blood sample is highly controllable. At least a part of casing 135 may be transparent and allow estimating a volume of the blood sample.

According to some embodiments of the invention, device 100 is advantageous over existed appliances in several aspects. First, device 100 does not require a power source such as a network connection or a battery, and is fully and exclusively operable by forces applied by a user. Second, device 100 may be cheaply constructed and thus be disposable. Furthermore, casing 135, cover 140, suction device 120 and pricking element 110 may be integrated into device 100 in a ready to use mode, such that device is sterilized and exhibits the partial vacuum. Device 100 may be packed in a loaded state in a bag ensuring its sterility. The loaded state may comprise the partial vacuum and a configuration of pricking element 110 allowing pricking the finger without any prior actions. Third, in its disposable embodiment, device 100 may be extremely simple to use in contrast to complicated existing instruments that require, e.g., lancet replacement and sterilization. Forth, device 100 allows a user to determine the amount or volume of the blood sample, either visually by looking through a transparent part of casing 135, or by adjusting the air pressure characterizing the partial vacuum, and the suction it applies upon the finger. Fifth, device 100 is characterized by enhanced safety, as a user does not deal with pricker 114 except for the actual pricking. Thus, device 100 embodies a simple, disposable device that would enable a patient to extract blood from his body, in a safe, reliable and without the need for help from professionals.

According to some embodiments of the invention, device 100 comprises: casing 135 comprising finger opening 130; suction device 120 arranged to produce a partial vacuum within casing 135; pricking element 110; and container 133. Pricking element 110 is arranged to prick a finger placed upon finger opening 130. Container 133 is arranged to collect blood trickling from the finger, such that the partial vacuum allows controlling the collected amount of blood.

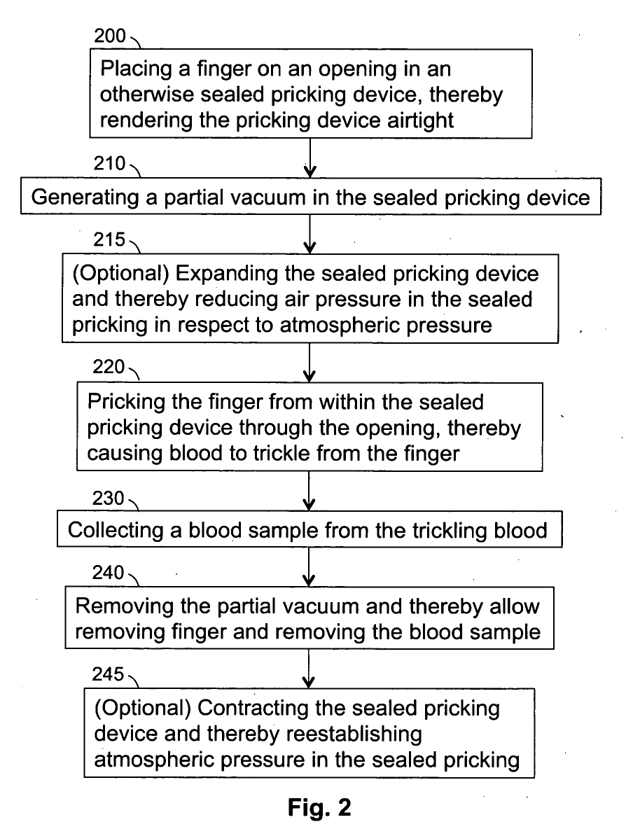

FIG. 2 is a high level schematic flowchart a method of extracting blood samples, according to some embodiments of the invention. The method comprises the following stages: placing a finger on an opening in an otherwise sealed pricking device (stage 200), thereby rendering the pricking device airtight; generating a partial vacuum in the sealed pricking device (stage 210); pricking the finger from within the sealed pricking device through the opening (stage 220), thereby causing blood to trickle from the finger; collecting a blood sample from the trickling blood (stage 230); and removing the partial vacuum (stage 240) and thereby allowing removing finger and removing the blood sample. The partial vacuum is selected such as to enhance blood trickling.

According to some embodiments of the invention, generating a partial vacuum in the sealed pricking device (stage 210) is carried out by expanding the sealed pricking device (stage 215) and thereby reducing air pressure in the sealed pricking in respect to atmospheric pressure.

According to some embodiments of the invention, removing the partial vacuum (stage 240) is carried out by contracting the sealed pricking device (stage 245) and thereby reestablishing atmospheric pressure in the sealed pricking.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

It is to be understood that the phraseology and terminology employed herein is not to be construed as limiting and are for descriptive purpose only.

The principles and uses of the teachings of the present invention may be better understood with reference to the accompanying description, figures and examples.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

It is to be understood that the terms “including”, “comprising”, “consisting” and grammatical variants thereof do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

The descriptions, examples, methods and materials presented in the claims and the specification are not to be construed as limiting but rather as illustrative only.

Meanings of technical and scientific terms used herein are to be commonly understood as by one of ordinary skill in the art to which the invention belongs, unless otherwise defined.

The present invention may be implemented in the testing or practice with methods and materials equivalent or similar to those described herein.

Any publications, including patents, patent applications and articles, referenced or mentioned in this specification are herein incorporated in their entirety into the specification, to the same extent as if each individual publication was specifically and individually indicated to be incorporated herein. In addition, citation or identification of any reference in the description of some embodiments of the invention shall not be construed as an admission that such reference is available as prior art to the present invention.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

What is claimed is:

1. A device for extracting blood samples comprising: a casing comprising a finger opening; a suction device arranged to produce a partial vacuum within the casing; a pricking element; and a container, wherein the pricking element is arranged to prick a finger placed upon the finger opening, and the container is arranged to collect blood trickling from the finger, such that the partial vacuum allows controlling the collected amount of blood.

2. The device of claim 1, further comprising a cover,
wherein the casing has a suction opening,
wherein the suction device is sealably connected to the suction opening,
wherein the container has a sealable sample opening,
wherein the cover is sealably connected to the casing such that the cover operably seals the sample opening,
wherein the container is posited below the finger opening,
wherein the casing is operably sealable by placing a finger upon the finger opening,
wherein the suction device is arranged to operably generate a partial vacuum in the sealed casing, the partial vacuum characterized by an air pressure lower than atmospheric pressure,
wherein the pricking element is arranged to prick the finger from within the casing through the finger opening such as to cause blood to trickle from the pricked finger,
wherein the partial vacuum enhances blood trickling from the pricked finger, and the air pressure is selected to enhance blood trickling,
wherein the container is arranged to hold a blood sample from the pricked finger, and
wherein the sample opening is arranged to allow the blood sample through upon releasing the partial vacuum.

3. The device of claim 1, wherein the suction device comprises a piston arranged to reduce air pressure, in the sealed casing, upon expansion.

4. The device of claim 2, wherein the piston is arranged to release the partial vacuum upon contraction.

5. The device of claim 2, wherein the suction device is further arranged to allow generating a predefined pressure, and the container is further arranged to allow removing the blood sample from the container through the sample opening upon the generation of the predefined pressure.

6. The device of claim 2, wherein the suction device is further arranged to allow controlling an amount of blood trickling from the pricked finger by regulating the air pressure and a time of using the device.

7. The device of claim 1, wherein the casing comprises a lower part comprising the container and the suction device and an upper part comprising the finger opening and the pricking element, the upper part is sealed upon the lower part.

8. The device of claim 1, wherein at least part of the casing is transparent and allows estimating a volume of the blood sample.

9. The device of claim 1, wherein the container is integrated within the casing.

10. The device of claim 1, wherein the pricking element comprises:
a lever pivotly connected to the casing, having an inner arm substantially inside the casing and an outer arm substantially outside the casing; and
a pricker mounted on the inner arm of the lever,
wherein the lever is arranged to bring the pricker in contact with the finger upon pushing the outer arm, and
wherein the pricker is arranged to prick the finger.

11. The device of claim 10, wherein the outer arm is connected to a lever spring arranged to remove the pricker from the finger upon releasing the outer arm.

12. The device of claim 2, wherein the casing, the cover, the suction device and the pricking element are integrated into the device in a ready to use mode, such that the device is sterilized and exhibits the partial vacuum.

13. The device of claim 1, arranged to operate exclusively by forces applied by a user.

14. The device of claim 1, further comprising a cap arranged to seal the finger opening.

15. The device of claim 14, wherein the cap is arranged keep the extracted blood sample in sterile conditions.

16. The device of claim 1, further comprising a medical testing means integrated within the device.

17. A method of extracting blood samples, comprising:
placing a finger on an opening in an otherwise sealed pricking device, thereby rendering the pricking device airtight;
generating a partial vacuum in the sealed pricking device;
pricking the finger from within the sealed pricking device through the opening, thereby causing blood to trickle from the finger;
collecting a blood sample from the trickling blood; and
removing the partial vacuum and thereby allowing removing the finger and removing the blood sample,
wherein the partial vacuum is selected such as to enhance blood trickling.

18. The method of claim 17, wherein generating a partial vacuum in the sealed pricking device is carried out by expanding the sealed pricking device and thereby reducing air pressure in the sealed pricking in respect to atmospheric pressure.

19. The method of claim 18, wherein removing the partial vacuum is carried out by contracting the sealed pricking device and thereby reestablishing atmospheric pressure in the sealed pricking.

20. A device for extracting blood samples, comprising:
a casing having a finger opening and a suction opening, the casing comprising a container having a sealable sample opening and posited below the finger opening;
a cover sealably connected to the casing such that the cover operably seals the sample opening;
a suction device sealably connected to the suction opening of the casing; and
a pricking element,
wherein the casing is operably sealable by placing a finger upon the finger opening,
wherein the suction device is arranged to operably generate a partial vacuum in the sealed casing, the partial vacuum characterized by an air pressure lower than atmospheric pressure,
wherein the pricking element is arranged to prick the finger from within the casing through the finger opening such as to cause blood to trickle from the pricked finger,
wherein the partial vacuum enhances blood trickling from the pricked finger, and the air pressure is selected to enhance blood trickling,
wherein the container is arranged to hold a blood sample from the pricked finger,
wherein the sample opening is arranged to allow the blood sample through upon releasing the partial vacuum,
wherein the suction device is further arranged to allow generating a predefined pressure, and the container is further arranged to allow removing the blood sample from the container through the sample opening upon the generation of the predefined pressure,
wherein the suction device is further arranged to allow controlling an amount of blood trickling from the pricked finger by regulating the air pressure and a time of using the device,
wherein at least part of the casing is transparent and allows estimating a volume of the blood sample,
wherein the casing, the cover, the suction device and the pricking element are integrated into the device in a ready to use mode, such that the device is sterilized and exhibits the partial vacuum, and
wherein the device is arranged to operate exclusively by forces applied by a user.
